(19) United States
(12) Patent Application Publication
(10) Pub. No.: US 2022/0362184 A1
(43) Pub. Date: Nov. 17, 2022

(54) GUT-PROTECTIVE COMPOSITIONS COMPRISING BOSWELLIC ACID

(71) Applicant: INVENTIA HEALTHCARE LIMITED, Mumbai, Maharashtra (IN)

(72) Inventors: Vaibhavi SHAH, Mumbai, Maharashtra (IN); Vijayendrakumar REDASANI, Mumbai, Maharashtra (IN); Shajahan ABDUL, Madurai, Tamil Nadu (IN); Vishal SHAH, Mumbai, Maharashtra (IN); Rajat SHAH, Mumbai, Maharashtra (IN)

(21) Appl. No.: 17/755,498

(22) PCT Filed: Dec. 23, 2019
(86) PCT No.: PCT/IB2019/061271

§ 371 Date: Apr. 29, 2022

(30) Foreign Application Priority Data
Nov. 5, 2019 (IN) 201921044814

(51) Int. Cl. A61K 31/19; A61K 36/324

(52) U.S. Cl. CPC A61K 31/19; A61K 36/324

(57) ABSTRACT

The invention describes a gut-protective composition comprising boswellic acid, at least one pH modifier, wherein the composition exhibits enhanced dissolution. The composition as described herein may be comprised of about 5 to 95% of boswellic acid. The said composition is stable and exhibits improved bioavailability. The invention also provides a process for preparation of the said composition, wherein boswellic acid and pH modifier are mixed well by optionally adding at least one or more excipient and processed to get granular powder. The composition may be further formulated into solid, semi solid or liquid dosage forms, for administration to human or animals. The compositions described herein exhibit gut-protective effect in conditions of colitis as well as microbial infections.

### FIELD OF THE INVENTION

The present invention relates to a gut-protective composition comprising boswellic acid and at least one pH modifier; wherein the composition exhibits enhanced dissolution. The compositions as described herein may be comprised of about 5 to 95% of boswellic acids as obtained from boswellic extract. The composition may optionally be comprised of one more excipient, which is acceptable for use in nutraceutical, pharmaceutical and food industry. The compositions comprising boswellic acid and pH modifier are stable and exhibit improved bioavailability. The invention further provides a process for preparation of the said composition, wherein boswellic acid and pH modifier are mixed well and granulated using suitable equipment. The granules may be further formulated into solid, semisolid or liquid dosage forms, for administration to humans and/or animals. The compositions described herein exhibit gut-protective effect in conditions of colitis as well as microbial infections.

### BACKGROUND OF THE INVENTION

Extract of ‘Boswellia Serrata’ (Indian Frankincense), contains components like volatile oils, resins, gum and terpenoids. The terpenoids include monoterpenes, diterpenes, triterpenes, tetracyclic triterpenic acids and pentacyclic triterpene acids. Pentacyclic triterpene acids (boswellic acids) include α-boswellic acid (ABA), β-boswellic acid (BBA), and γ-boswellic acid, and their acetyl and/or keto derivatives including 3-acetyl-β-boswellic acid (ABBA), 11-keto-β-boswellic acid (KBA), 3-acetyl-α-boswellic acid (AABA) and 3-acetyl-11-keto-β-boswellic acid (AKBA).

Boswellic acids are effective anti-inflammatory and analgesic agents, having better tolerance and lesser side effects compared to NSAIDs. Boswellic acids have been reported to be useful in treating certain tumors, and cancers such as leukemia and breast cancer. Boswellic acids also demonstrate therapeutic improvements in brain related disorders like peritumoral oedema, and attention-deficit hyperactivity disorder (ADHD), and are believed to enhance cognition, memory, learning and communication. Other therapeutic uses of boswellic acids related to their antifungal, anti-microbial, psoriasis, anti-hyperlipidemic, anti-artherosclerotic, antioxidant, immunomodulatory, autoimmune encephalomyelitis and hepatitis-C inhibition effects have also been evaluated.

Even though boswellic acid is known for number of its therapeutic uses, major impediment in the development of boswellic acids formulation is their poor pharmacokinetics. Boswellia extracts contain at least 15 triterpene acids, out of which AKBA, KBA and BBA are most potent with respect to therapeutic activity. These boswellic acids exhibit poor oral bioavailability due to the high lipophilicity (log P=7.0-10.3) and poor aqueous solubility, resulting in poor rate and extent of absorption.

Prior art describes various delivery systems of boswellic acids, mainly focused on their efforts to improve dissolution and bioavailability.

Anshuman Bharadwaj et al. (Indian Journal of Natural Products and Resources. 2016, Vol 7(1), 9-18) and Kunal Detholia et al. (IOSR Journal Of Pharmacy, 2017, Vol. 7(12) 75-83) describe SMEDDS (self-microemulsifying drug delivery system) of Boswellia serrata extracts (BSE) to enhance aqueous solubility, better absorption profile, enhance bioavailability and improve pharmacokinetics to ensure effective anti-inflammatory activity of boswellic acids. The SMEDDs use various components like oils, surfactants and co-surfactants. The SMEDDS use oils such as castor oil, caprylic/capric triglycerides (CCTG), ethyl oleate, Capmul MCM® oil, surfactants such as Tween® 80, Cremophor® EL, Cremophor® RH-40, Labrasol®, Labrafil® M1944CS, Lutrol® E-300 and Transcutol® P, and Acrysol EL135®, and co-surfactants such as polyethylene glycol 400 and Acconon® MC8, and processes like vortexing and sonication.

The BSE loaded SMEDDS formulation F1 A of Anshuman Bharadwaj et al. showed release of 56.84%, 60% and 53.68% of AKBA after 60 min, in water, HCl buffer pH 1.2, and phosphate buffer pH 6.8 respectively. In in-vitro drug release studies, drug release in 0.1N HCl from the SMEDDS formulation F1A was found to be more than 90% after 120 minutes which was significantly higher as compared with that of plain BSE.

Kunal Detholia et al. reported a dissolution study using the USP-II apparatus using 900 ml of 0.1N HCl as dissolution media and 50 rpm as the paddle rotation speed. BSE-SMEDDS tablet released more than 80% of active constituents within 30 minutes.

No bioavailability studies were reported in Anshuman Bharadwaj et al. and Kunal Detholia et al.

F. Franceschi et al. (European Review for Medical and Pharmacological Sciences, 2016; 20: 4156-4161), T. Ferrara et al. (European Review for Medical and Pharmacological Sciences, 2015; 19: 3757-3762) and Jan Husch et al. (Fitoterapia 2013; 84:89-98) disclose a lecithin-based phytosome delivery form of Boswellia serrata extract to improve the bioavailability of the said extract. The said extract and soy lecithin are in 1:1 ratio, with microcrystalline cellulose being added to improve the physical state and to standardize the product. In Jan Husch et al., animal studies were conducted in female albino Wistar rats. Phytosome compositions with a dosage equiweight to and a dosage equimolar to, 240 mg/kg of boswellia extract (88.28 mg of extract contained 7.77 mg of AKBA), were administered to the rats. For phytosomes administered at equimolar doses, the phytosomes showed a mean Cₘₐₓ of about 621 ng/ml, a mean AUC₀₋ₗₐₛₜ of about 2619 ng/ml and a mean Tₘₐₓ of about 4.17 hours.

US Patent Publication No. 2014/0039031 is directed to compositions and methods for formulating a pharmaceutical dosage form comprising acetyl-11-keto-β-boswellic acid, diindolylmethane, or curcumin with one or more pharmaceutically acceptable excipients, for enhanced solubility to increase bioavailability and improve therapeutic efficacy. The said compositions are thermo-kinetically compounded and further processed using processes like hot melt extrusion, melt granulation, compression molding, tablet compression, capsule filling, film-coating, or injection molding. Formulations were subjected to a bioavailability study in male Sprague-Dawley rats, at a dose equivalent to 50 mg/kg AKBA. The compositions demonstrated an AUC of AKBA ranging from about 1.45 to about 12.55 μg-hr/ml, a Cₘₐₓ of AKBA ranging from about 0.9 to about 4.17 μg/ml, and a Tₘₐₓ of AKBA ranging from about 0.61 hours to about 2.13 hours.

Amruta Tambe et al. (Journal of Drug Delivery Science and Technology 2018, 44, 172-180) enhanced solubility and dissolution of boswellic acids by solid dispersion technique using poloxamer 188 and 407 as hydrophilic polymeric carriers. The solid dispersions were prepared using the kneading method and the solvent evaporation method, with boswellic acids and poloxamer in ratios 1:1 and 1:2 and ethanol as a solvent.

U.S. patent Ser. No. 10/286,023 relates to non-acidic Boswellia low polar gum resin extract fraction in combination with biological agents such as Boswellia extract/Boswellic acids or curcumin, for enhancing the bioavailability. The invention also provides the method of enhancing the bioavailability of biological agents by using oily fractions derived from Boswellia.

US patent application 20190175678 describes a synergistic anti-inflammatory composition comprising an extract selectively enriched in 3-O-acetyl-11-keto-O-boswellic acid (AKBA) derived from Boswellia serrata; and a Boswellia serrata non-acidic resin extract (BNRE). The compositions can be used to prevent, control and treat inflammation and several inflammatory related diseases including asthma, osteoarthritis, rheumatoid arthritis, and/or endothelial dysfunction.

Prior art formulation systems are prepared as SMEDDS, phytosomes, and solid dispersions, using excipients like lipids, surfactants, plasticizers, organic/aqueous solvents. Some of the references also make use of combination of active with oily fraction or low polarity fraction derived from Boswellia to enhance bioavailability or to enhance therapeutic effect of the active in the body.

There exists a need to develop alternate formulation options, using excipients from natural source, in order to enhance the dissolution of compositions comprising boswellic acids and render those suitable for nutraceutical and food applications. The need also exists for developing the compositions from natural origin, which will have gut-protective effects and can be used as substitute for NASID (non-steroidal anti-inflammatory drugs) which are known for their side effects in the form of gastrointestinal (GI) complications including abdominal discomfort, bleeding resulting in life threatening ulceration and GI wall perforations.

The present invention provides a gut-protective composition comprising boswellic acid and at least one pH modifier, wherein the composition is stable and exhibits enhanced dissolution. The pH modifier may be obtained from synthetic or natural sources, preferably natural source and thus useful and safe for nutraceutical, food as well as pharmaceutical applications. The invention also provides process for the preparation, which is simple, economic and employs commonly used equipment, thus making it industrially useful and suitable for administration to humans and/or animals.

### OBJECTS OF THE INVENTION

An object of the present invention is to provide a gut-protective composition comprising boswellic acid and at least one pH modifier, wherein the composition exhibits enhanced dissolution.

Another object of the present invention is to provide a stable composition comprising 5 to 95% boswellic acids and at least one pH modifier, wherein the boswellic acid exhibits improved bioavailability.

One more objective of the present invention is to provide a boswellic acid composition comprising a pH modifier which may be obtained from natural and/or synthetic source, preferably from natural source.

Yet another object of the present invention is to provide boswellic acid compositions which may optionally contain one more excipient which is acceptable in pharmaceutical, nutraceutical and food industry.

One important objective of the present invention is to provide the compositions comprising boswellic acids selected from the group of, but not limited to α-boswellic acid (ABA), β-boswellic acid (BBA), and γ-boswellic acid, its acetyl or keto derivative including 3-acetyl-β-boswellic acid (ABBA), 11-keto-β-boswellic acid (KBA), 3-acetyl-α-boswellic acid (AABA) and 3-acetyl-11-keto-β-boswellic acid (AKBA), a pharmaceutically or nutraceutically acceptable salt, an active metabolite, a polymorph, a solvate, a hydrate, an enantiomer, an optical isomer, a tautomer or a racemic mixture thereof. Boswellic acids are obtained from Boswellia extract.

Yet another objective of the present invention is to provide the compositions comprising 5 to 95% of boswellic acids by weight of the composition.

Another objective of the present invention is to provide the compositions comprising 10 to 80% of a pH modifier by weight of the composition.

Still one more objective of the inventions is to provide a process for preparation, wherein boswellic acid and pH modifier are mixed well by optionally adding one or more excipients and granulated using suitable equipment. The granular powder composition may be further formulated into solid, semisolid or liquid dosage forms, for administration to human and/or animals. The compositions as described herein may be optionally comprised of about 5 to 20% of an excipient by weight of the composition.

One object of the present invention is to provide boswellic acid compositions which exhibit gut-protective effect in conditions of colitis and microbial infections.

Another important objective of the present invention is to provide boswellic acid compositions which exhibit protective effect in colitis like conditions and the microbial infections. The administration of boswellic acids composition indicated better regeneration of damaged tissues in process of combating colitis and maintaining gut-health.

### DETAILED DESCRIPTION OF THE INVENTION

Surprisingly it has been found that the compositions of the present invention comprising boswellic acid and at least one pH modifier, exhibit enhanced dissolution as compared to unformulated boswellic acid. The composition is also found to exhibit gut-protective effect, especially in case of colitis conditions and the microbial infections. Further, the compositions comprising boswellic acids also exhibit improved bioavailability.

The present invention makes use of extract of Boswellia serrata containing at least 5% boswellic acid. The term ‘boswellia extract” as used herein means the extract of the oleo gum resin of Boswellia serrata.

The term ‘gut-protective effect’ as used herein means the effect of compositions comprising boswellic acids in protecting, improving and maintaining healthy gut related gastro-intestinal functions. Gut health comprises a healthy upper and lower GI tract, which is free of complaints such as functional dyspepsia, colitis, bowel syndrome and comprise flatulence, bloating, regurgitation, heartburn, nausea, vomiting, constipation, diarrhoea, food intolerance, incontinence, abdominal pain and cramps, loss of appetite, weight loss and blood in stools. Sound gut health covers multiple positive aspects of the gastrointestinal (GI) tract, such as the effective digestion and absorption of food, the absence of GI illness, normal and stable intestinal microbiota, effective immune status and a state of well-being. The compositions as described herein helps for recovery from colitis and also has anti-microbial effect, especially against Helicobacterium pylori (H. pylori) which is a main cause for colitis and colon cancers.

The term “boswellic acid” is used herein to mean pentacyclic triterpene acid selected from the group of, but not limited to α-boswellic acid (ABA), β-boswellic acid (BBA), and γ-boswellic acid, or its acetyl and/or keto derivative including 3-acetyl-β-boswellic acid (ABBA), 11-keto-β-boswellic acid (KBA), 3-acetyl-α-boswellic acid (AABA) and 3-acetyl-11-keto-β-boswellic acid (AKBA), including their salt, derivative, active metabolite, a polymorph, a solvate, a hydrate, an enantiomer, an optical isomer, a tautomer and a racemic mixture thereof.

As per one more embodiment, the boswellic extract used in the present invention may comprise boswellic acids—α-boswellic acid (ABA), β-boswellic acid (BBA), 3-acetyl-β-boswellic acid (ABBA), 11-keto-β-boswellic acid (KBA), 3-acetyl-α-boswellic acid (AABA) and 3-acetyl-11-keto-β-boswellic acid (AKBA), cumulatively at a concentration of about 30% to about 85% by weight of the extract, preferably about 50% to about 80% by weight of the extract.

As per one more embodiment, the boswellic compositions of the present invention may comprise at least about 5% by weight of acetyl-11-keto-β-boswellic acid (AKBA), more preferably from about 10% to about 80%, more preferably from about 15% to about 70% and most preferably from about 20% to about 50%, by weight of acetyl-11-keto-β-boswellic acid (AKBA).

The compositions of the present invention may comprise at least about 50% by weight of acetyl-11-keto-β-boswellic acid (AKBA), preferably from about 60% to about 90%, more preferably from about 70% to about 90%, by weight of acetyl-11-keto-β-boswellic acid (AKBA).

The compositions of the present invention may comprise at least about 5% by weight of β-boswellic acid (BBA), more preferably at least about 10% by weight of the composition. The compositions of the present invention may comprise β-boswellic acid (BBA) in concentrations of about 5% to about 50%, preferably about 5% to about 40% by weight of the composition.

The compositions may comprise at least about 0.5% by weight of 11-keto-β-boswellic acid (KBA), more preferably at least about 2% by weight of the extract. The boswellic extract used in the invention may comprise 11-keto-β-boswellic acid (KBA) in concentrations of about 1% to about 20% by weight of the composition.

The boswellia extract may be present in concentrations from 5% to 95% by weight of the composition. The compositions of the present invention may be comprised of about 5 to 95% by weight of the boswellic acids.

The term ‘pH modifier’ as used herein means the excipient which changes pH of the microenvironment of the active (in this case boswellic acids), when it is used as part of the composition. pH modifier selected in the composition of the invention is having alkaline properties, which helps to change pH of the composition from acidic to alkaline, thus modifying the pH of the system. Use of pH modifier changes the pH of microenvironment of the composition and helps to improve dissolution.

As per one embodiment of the invention, pH modifier may be selected from the group of, but not limited to alkaline agents, amino sugars, alkali metal salts, alkaline earth metal salts, metal oxide, organic bases, organic basic salts, inorganic bases, inorganic basic salts, conjugate bases of weak organic acids, and mixtures thereof.

The pH modifier may be selected from the group of, but not limited to meglumine, eglumine, N-methyl glucamine, ammonium hydroxide, magnesium oxide, aluminum oxide, magnesium hydroxide, calcium hydroxide, sodium hydroxide, sodium citrate dihydrate, sodium lauryl sulphate, sodium acetate, potassium hydroxide, aluminum hydroxide, potassium carbonate, sodium bicarbonate, calcium carbonate, a phosphate salt such as calcium hydrogen phosphate, monobasic sodium phosphate, dibasic sodium phosphate, monobasic potassium phosphate, dibasic potassium phosphate, monobasic calcium phosphate, dibasic calcium phosphate, monobasic magnesium phosphate, dibasic magnesium phosphate, monobasic ammonium phosphate, dibasic ammonium phosphate and mixtures thereof. Preferably pH modifier is selected from natural source.

As per one embodiment of the invention, the pH modifier may be present in concentration of about 10% to about 80% by weight of the composition.

According to one embodiment of the invention, the composition of the present invention may be comprised of boswellic acid and pH modifier at weight ratio of 1:0.1 to 1:10, preferably at weight ratio of 1:0.2 to 1:8.

As per one embodiment of the invention, the compositions may optionally be comprised of one more excipient selected from diluents, carriers, binders, disintegrants, lubricants, glidants, surfactants, solubilizers, wetting agents, carriers, vehicles, stabilizers, buffers, preservatives, sorbents, antioxidants, complexing agents, viscosity enhancers, plasticizers, coating materials, sweeteners, colors, and flavors.

The compositions may be optionally comprised of one excipient selected from the group of diluents known in the art, but not limited to microcrystalline cellulose, silicified microcrystalline, cellulose, powdered cellulose, microfine cellulose, corn starch, rice bran extract, calcium phosphate, dibasic calcium phosphate, tribasic calcium phosphate, calcium sulfate, or mixtures thereof. The diluents may also be selected from glucose, lactose, sucrose, dextrose, fructose, compressible sugar, or mixtures thereof.

The diluents may be optionally present in concentrations from 1% to 50% by weight of the composition. Preferably, the diluents may be optionally present in concentrations from 3% to 40% by weight of the composition.

The binders may be selected from the group of cellulose derivatives such as hydroxypropylmethyl cellulose (HPMC), hydroxypropyl cellulose (HPC), ethylcellulose, carboxymethylcellulose (CMC), sodium CMC, potassium CMC, calcium CMC, methylcellulose, hydroxyethyl cellulose (HEC), microcrystalline cellulose; polyvinylpyrrolidone (PVP), vinyl pyrrolidone-vinyl acetate copolymer, polyvinyl alcohol, starch, carbomer, gums like xanthan gum, guar gum, acacia, locust bean gum, alginates, or mixtures thereof.

The binders may be present in concentrations from 1% to 50% by weight of the composition. Preferably, the binders may be present in concentrations from 5% to 25% by weight of the composition.

The disintegrants may be selected from sodium starch glycolate, crospovidone, croscarmellose sodium, croscarmellose calcium, croscarmellose potassium, sodium carbonate, sodium hydrogen carbonate, calcium carbonate, starch, starch 1500, modified starch, pregelatinized starch, crosslinked carboxymethyl starch, sodium hydrogen carbonate, hydroxypropyl cellulose, sodium carboxymethylcellulose or mixtures thereof.

The disintegrants may be used in concentrations from 1% to 25% by weight of the composition. Preferably, the disintegrants may be present in concentrations from 5% to 20% by weight of the composition.

The lubricants may be selected from magnesium stearate, calcium stearate, sodium benzoate, talc, or mixtures thereof. The lubricants may be present in concentrations from 0.25% to 5% by weight of the composition.

The glidants may be selected from suitable glidants known in the art. An example of a suitable, acceptable glidant is colloidal silicon dioxide. The glidants may be present in concentrations from 0.1% to 10% by weight of the composition.

The compositions comprising boswellic acids can be manufactured using processes like wet granulation, coating, layering, spray-drying, freeze-drying, dissolving, dispersing, suspending, or emulsifying which involve the use of non-aqueous solvents and/or aqueous solvents.

Alternatively, the compositions can be manufactured using solvent-free processes like slugging, dry granulation, direct compression, hot-melt extrusion, hot-melt granulation, hot-melt solidification, extrusion, spheronization, compaction, compression, compression coating, powder coating, layering, spray-drying, freeze-drying, or spray chilling.

As per one embodiment of this invention, boswellic acids are mixed with pH modifier and mixed well. The mixture may be optionally further added with one more excipient, which is acceptable for use in nutraceutical, pharmaceutical and food industry. The mixture may be processed further using suitable equipment to get the granular powder composition.

For oral administration, the compositions can be formulated in the form of tablets, effervescent tablets, orally-disintegrating tablets, mouth dissolving tablets, sublingual tablets, buccal tablets, capsules, pills, chewing gums, films, powders, granules, beads, pellets, lozenges, pastilles, soft-gels, semi-solid, pastes, syrups, elixirs, solutions, suspensions, dispersions, emulsions and the like.

The compositions of the invention can be used for oral, peroral, enteral, topical, transdermal, parenteral, ophthalmic, rectal, and vaginal administration.

The composition of the present invention, comprising boswellic acid and at least one pH modifier, is stable over shelf life. The compositions were analyzed for the assay using HPLC method during stability study.

The compositions were analyzed for their dissolution using 900 ml of purified water containing 0.5% sodium lauryl sulphate, USP apparatus II (paddle) at 100 revolutions per minute, 37° C.

The compositions of the present invention exhibit a cumulative dissolution of not less than 80% by weight of boswellic acid in 15 minutes and complete release within 30 minutes.

The pharmacokinetics of the composition is studied following a single oral administration in male Sprague Dawley rats to check maximum plasma concentrations (Cₘₐₓ), AUC₀₋₂₄, and time to reach maximum plasma concentrations (Tₘₐₓ) values, to understand rate and extent of absorption of boswellic acid into the blood, when compared to boswellia extract.

The compositions comprising boswellic acids are also evaluated for their gut-protective effect using Dextran Sodium Sulphate (2.5% w/v) induced colitis in BALB/c mice. Body weights of mice were recorded during study period, macroscopic examination was carried out for all terminal sacrificed, moribund and found dead animals. Colon and rectum were collected, and histopathology was performed. The composition comprising boswellic acid was also evaluated for gut-protective effect through antimicrobial activity against Helicobacter pylori strains using Micro-broth dilution method. The anti-bacterial minimum inhibitory concentration (MIC) was checked for the composition against Amoxycillin.

The invention is now illustrated with non-limiting examples.

### Example 1

Boswellia extract containing 92.44% AKBA, and pH modifiers selected from N-methyl glucamine and magnesium oxide, in different ratios, were sifted through 30 mesh ASTM and mixed for 10 minutes to give powder compositions. The details of the said powder compositions are given in Table 1.

TABLE 1
Details of the powder compositions of Example 1

| | 1a | 1b | 1c | 1d | 1e | 1f |
|---|---|---|---|---|---|---|
| Composition | Quantities in mg | | | | | |
| Boswellia extract | 21.6* | 21.6* | 21.6* | 21.6* | 21.6* | 21.6* |
| (92.44% AKBA) | | | | | | |
| Meglumine | — | 10 | — | — | 50 | 100 |
| Magnesium Oxide | 5 | — | 10 | 20 | — | — |
| Ratio of AKBA: | 1:0.25 | 1:0.5 | 1:0.5 | 1:1 | 1:2.5 | 1:5 |
| pH modifier | | | | | | |

*21.6 mg of Boswellia extract contains 20 mg of AKBA

Dissolution Studies:

Boswellia extract containing 92.44% AKBA, and the powder compositions of Example 1, all equivalent to 50 mg of AKBA, were analyzed for dissolution of AKBA using 900 ml of purified water containing 0.5% sodium lauryl sulphate, USP apparatus II (paddle) at 100 revolutions per minute, 37° C. Table 2 gives the results of the dissolution study.

TABLE 2
Dissolution of the Boswellia extract (92.44% AKBA)
and the powder compositions of examples 1a to 1f

| | % Cumulative Dissolution Profile | | | | | | |
|---|---|---|---|---|---|---|---|
| Time | Boswellia | | | | | | |
| (min) | extract | 1a | 1b | 1c | 1d | 1e | 1f |
| 15 | 57% | 90% | 70% | 95% | 94% | 99% | 99% |
| 30 | 80% | 100% | 91% | 101% | 100% | 101% | 101% |
| 45 | 88% | 100% | 96% | 101% | 101% | 102% | 102% |
| 60 | 93% | 101% | 97% | 101% | 101% | 102% | 103% |

*min = minutes

The dissolution data in Table 2 shows:
At 15 minutes, the powder compositions of examples 1a to 1f exhibit a dissolution of not less than 70% when compared to a mere 57% for unformulated boswellic acids.
At 30 minutes, the powder compositions of examples 1a to 1f exhibit a dissolution of not less than 90% when compared to 80% for unformulated boswellic acids.

The dissolution results in Table 2 clearly indicate that powder compositions of the present invention exhibit a significantly higher rate and extent of dissolution, when compared tounformulated boswellic acids.

### Example 2

Boswellia extract containing 92.44% AKBA (23.28% w/w of composition), N-methyl glucamine (52.81% w/w of composition), poloxamer 188 (21.91% w/w of composition), and colloidal silica (0.5% w/w of composition) were sifted through 30 mesh ASTM and mixed for 10 minutes to give a powder composition. The powder composition was hot melt extruded under the conditions mentioned in Table 3, cooled and milled in a co-mill (1 mm sieve). Colloidal silicon dioxide (1.5% w/w of composition) was sifted through 30 mesh ASTM. The granules and colloidal silicon dioxide were co-sifted through 30# and mixed, to give the granular powder composition.

### Example 3

Boswellia extract (containing 31.51% AKBA) (80.64% w/w of composition), N-methyl glucamine (14.20% w/w of composition), poloxamer 188 (4.4% w/w of composition), and colloidal silica (0.5% w/w of composition) were sifted through 30 mesh ASTM and mixed for 10 minutes to give a mixture. The mixture was hot melt extruded under the conditions mentioned in Table 3, cooled and milled in a co-mill (1 mm sieve). Colloidal silicon dioxide (0.26% w/w of composition) was sifted through 30 mesh ASTM. The granules and colloidal silicon dioxide were co-sifted through 30# and mixed, to give the granular powder composition.

TABLE 3
Hot melt extruder conditions for processing of Examples 2 and 3
Temperature of Chambers

| Chamber | Temperature (° C.) |
|---|---|
| 2 | 30 |
| 3 | 60 |
| 4 | 110 |
| 5 | 110 |
| 6 | 110 |
| 7 | 110 |
| 8 | 110 |
| Die Zone | 120 |

Extruder rpm = 75
Feeder rpm = 150

Dissolution Studies:

Boswellia extract containing 92.44% AKBA, powder composition of Example 2, extrudate composition of Example 2, Boswellia extract containing 31.51% AKBA, and extrudate composition of Example 3, all equivalent to 50 mg of AKBA, were analyzed for dissolution of AKBA at conditions mentioned in Example 1. Table 4 gives the results of the dissolution study.

TABLE 4
Dissolution of Boswellia Extracts, powder composition
and extrudate compositions of examples 2 and 3

| | % Cumulative Dissolution Profile | | | | |
|---|---|---|---|---|---|
| | Boswellia | | | Boswellia | |
| | extract | Powder | Composition | extract | Composition |
| Time | (92.44% | mixture of | of | (31.51% | of |
| (min) | AKBA) | Example 2 | Example 2 | AKBA) | Example 3 |
| 15 | 57% | 96% | 106% | 46% | 80% |
| 30 | 80% | 106% | 107% | 71% | 92% |
| 45 | 88% | 106% | — | 86% | 98% |
| 60 | 93% | 107% | — | 91% | 102% |

*min = minutes

The dissolution data in Table 4 shows:
At 15 minutes, the powder composition of example 2, and extrudate compositions of example 2 and 3, exhibit a dissolution of not less than 80% when compared to a mere 46% to 57% for unformulated boswellic acids.
At 30 minutes, the powder composition of example 2, and extrudate compositions of example 2 and 3, exhibit a dissolution of not less than 90% when compared to a mere 70%-80% for unformulated boswellic acids.

The dissolution results in Table 4 clearly indicate that the powder and extrudate compositions of the present invention, exhibit a significantly higher rate and extent of dissolution, when compared to unformulated boswellic acids.

### Example 4

Boswellia extract (containing 30% AKBA) (83.33% w/w of composition), rice bran extract (8.33% w/w of composition) and magnesium oxide (8.34% w/w of composition) were sifted through 60 mesh ASTM and mixed for 10 minutes to give a mixture. The mixture was subjected to twin screw extruder under the conditions mentioned in Table 5. The granules were sifted through 80# ASTM to give the granular powder composition. Three reproducible batches of this composition were prepared and analyzed for dissolution.

TABLE 5
Twin screw extruder conditions for processing of Example 4
Temperature of Chambers

| Chamber | Temperature (° C.) |
|---|---|
| 2 | 20 |
| 3 | 20 |
| 4 | 20 |
| 5 | 20 |
| 6 | 20 |
| 7 | 20 |
| 8 | 20 |
| Die Zone | 20 |

Extruder rpm = 150
Feeder rpm = 25

Dissolution Studies:

The composition of Example 4 was analyzed for dissolution of AKBA at conditions mentioned in Example 1. HPLC method was used for this analysis, using Agilent XDB C18 column and Acetonitrile:Water:Glacial acetic acid (900:100:0.1) as mobile phase. Injection volume was 2 □L and detection was carried out 254 nm. Table 6 gives the results of the dissolution study.

TABLE 6
Dissolution of extrudate compositions of example 4

| | % Cumulative Dissolution Profile | | | |
|---|---|---|---|---|
| Time | Boswellia extract | Batch 1 of | Batch 2 of | Batch 3 of |
| (minutes) | (30% AKBA) | Example 4 | Example 4 | Example 4 |
| 15 | 46% | 100 | 101 | 100 |
| 30 | 71% | 103 | 102 | 103 |
| 45 | 86% | 103 | 103 | 103 |
| 60 | 91% | 104 | 103 | 104 |

The dissolution data in Table 6 shows:
At 15 minutes, all three reproducible batches of composition of example 4, exhibit a dissolution of not less than 90% when compared to a mere 57% for unformulated boswellic acid.
At 30 minutes, all three reproducible batches of composition of example 4, exhibit a dissolution about 100% when compared to a mere 80% for unformulated boswellic acid.

The dissolution results in Table 6 clearly indicate that all three batches of the extrudate composition of the present invention, exhibit significantly higher rate and extent of dissolution, when compared to unformulated boswellic acid (Boswellia extract).

### Assay of Composition of Example 4

HPLC method was used for assay of the composition, using Kromacil C18 column and Acetonitrile:Water:Glacial acetic acid (900:100:0.1) as mobile phase. Detection was carried out at 254 nm & 210 nm (Dual Wavelength). Injection volume was 20 mL and run time was 45 minutes. Column temperature was 25° C.

TABLE 7
Assay Values for Boswellic acids content in the composition

| Composition of | Boswellic acids content | | |
|---|---|---|---|
| Example 4 | AKBA | KBA | BBA |
| Batch 1 | 25.99 | 3.84 | 10.19 |
| Batch 2 | 25.95 | 3.83 | 10.14 |
| Batch 3 | 25.98 | 3.82 | 10.15 |

Table 7 indicates that all three batches have specific percentage contents of boswellic acids—AKBA, KBA and BBA, as per the specification of the Boswellia extract.

Stability Study of Boswellic Acid Compositions:

Three reproducible batches of Boswellic acid composition of Example 4 were kept on stability in Alu-Alu pouch packed in blue drum at 40° C./75% relative humidity conditions for 3 months. Dissolution of the composition was checked in terms of % average of AKBA (3-acetyl-11-keto-ß-boswellic acid) at initial time point and after completion of 1, 2 and 3 months, by using the method described in Example 1. Assay of boswellic acids (AKBA, KBA and BBA) was also checked at initial time point and at regular interval of 1 month, till the end of 3 months.

TABLE 8
Stability study of Boswellic acid
composition in terms of Dissolution

| | |
|---|---|
| Batch | Dissolution (% Avg-AKBA) |

| No. | Month | 15 min | 30 min | 45 min | 60 min |
|---|---|---|---|---|---|
| 1 | Initial | 103 | 103 | 103 | 104 |
| | 1 | 105 | 111 | 108 | 107 |
| | 2 | 99 | 108 | 103 | 109 |
| | 3 | 100 | 102 | 101 | 101 |
| 2 | Initial | 101 | 102 | 103 | 104 |
| | 1 | 104 | 110 | 106 | 105 |
| | 2 | 107 | 113 | 104 | 108 |
| | 3 | 95 | 94 | 96 | 102 |
| 3 | Initial | 100 | 103 | 103 | 104 |
| | 1 | 106 | 108 | 108 | 111 |
| | 2 | 96 | 111 | 107 | 104 |
| | 3 | 98 | 98 | 98 | 100 |

TABLE 9
Stability Study in terms of % Assay of Boswellic acids

| | | % Assay | | | |
|---|---|---|---|---|---|
| | | 3-acetyl-11-keto-β- | | 11-keto-β- | β-boswellic |
| Batch | | boswellic acid | | boswellic acid | acid |
| No. | Month | w/w | Assay | w/w | w/w |
| 1 | Initial | 25.99 | 103.96 | 3.84 | 10.19 |
| | 1 | 25.56 | 102.24 | 3.76 | 10.08 |
| | 2 | 26.36 | 105.44 | 3.86 | 9.91 |
| | 3 | 25.86 | 103.44 | 3.66 | 9.92 |
| 2 | Initial | 25.95 | 103.8 | 3.83 | 10.14 |
| | 1 | 25.89 | 103.56 | 3.8 | 10.18 |
| | 2 | 26.31 | 105.24 | 3.86 | 9.95 |
| | 3 | 25.83 | 103.32 | 3.62 | 9.71 |
| 3 | Initial | 25.98 | 103.92 | 3.82 | 10.15 |
| | 1 | 25.05 | 100.2 | 3.75 | 10 |
| | 2 | 25.71 | 102.84 | 3.86 | 9.91 |
| | 3 | 25.7 | 102.8 | 3.67 | 9.9 |

It was observed that during stability study of boswellic acid composition,
the dissolution of all 3 batches in terms of % content of AKBA at all time points till 3 months was same as of the initial point.
Also, the assay of all three boswellic acids remained constant over the period of 3 months.

Thus the composition comprising boswellic acids is found to be stable.

### Example 5: Pharmacokinetic Studies

The pharmacokinetics of AKBA was studied following a single oral administration of the extrudate composition of Example 2 (containing Boswellia Extract containing 92.44% AKBA), in comparison with boswellia extract containing 92.44% AKBA alone. The study was conducted in male Sprague Dawley rats. Animals were orally administered with composition of Example 2 and Boswellia Extract 92.44% AKBA, each at a dose equivalent to 20 mg of AKBA/kg of body weight. Blood samples were collected at 0 (pre dose), 1, 2, 3, 4, 6, 8, 12, and 24 hours. Plasma samples were analyzed for Cₘₐₓ, AUC and Tₘₐₓ of AKBA. Table 5 gives the results of the pharmacokinetic data.

TABLE 10
Pharmacokinetic data

| | Boswellia Extract | Composition of Ex. 2 |
|---|---|---|
| Parameter | Mean (±SD) | Mean (±SD) |
| AKBA Cₘₐₓ (ng/ml) | 885.02 ± 284.73 | 2678.7 ± 1400.8 |
| AKBA AUC₀₋₂₄ (h*ng/ml) | 3761.02 ± 1592.08 | 9141.9 ± 5164.36 |
| AKBA Tₘₐₓ (hours) | 3.25 ± 0.71 | 1.13 ± 0.35 |

*Ex. = Example,
ng = nanogram,
ml = milliliter;
h = hour

The in-vivo data in Table 10 shows that on oral administration
Composition of the present invention exhibited a Cₘₐₓ of about 2678.7 ng/ml, which is about 3 times higher the Cₘₐₓ of boswellia extract (885.02 ng/ml) Composition of the present invention exhibited a AUC₀₋₂₄ of about 9141.9 ng/ml, which is about 2.5 times higher the AUC₀₋₂₄ of boswellia extract (3761.02 ng/ml)
Composition of the present invention was able to attain the maximum plasma concentrations in just about 1.1 hours (Tₘₐₓ), when compared to the extract which took about 3.3 hours (Tₘₐₓ)

The maximum plasma concentrations (Cₘₐₓ), AUC₀₋₂₄, and time to reach maximum plasma concentrations (Tₘₐₓ) values, clearly indicate that the compositions of the present invention exhibit higher rate and extent of absorption of boswellic acid into the blood, and an early onset of action, when compared to unformulated boswellic acids as obtained from Boswellia extract.

Thus, the present invention provides compositions comprising boswellic acid and at least one pH modifier, wherein the boswellic acid exhibits enhanced dissolution. The present invention further provides compositions comprising boswellic acid, wherein the boswellic acid exhibits improved bioavailability, with an early onset of action.

### Example 6: Evaluation of Gut-Protective Effect of the Composition Comprising Boswellic Acids

The gut-protective effect of composition comprising boswellic acids was evaluated using Dextran Sodium Sulphate (2.5% w/v) induced colitis in BALB/c mice. Dextran Sodium Sulphate (DSS) (36000 to 50000 Daltons molecular weight) was added in autoclaved drinking water and administered to the animals over 10 days for inducing disease condition. Group 2 served as disease control. Groups 3, 4 and 5 were treated with Test Item 1 (G3), Test Item 2 (G4) and Reference (Sulfasalazine—G5), respectively. Concurrent vehicle control group (G1) was also maintained.

Onset of colitis symptoms was monitored by measuring percent body weight, stool consistency (SC) and fecal occult blood (FOB). During the study period (10 days), Disease Activity Index (DAI) was calculated from scores of percent body weight, SC and FOB and were reported. Treatment was continued along with the DSS administration in drinking water up to 10 days by oral administration of Test and Reference Item as follows:
Group 1—Normal control (no DSS & no treatment)
Group 2—Disease control (only DSS, no treatment)
Group 3—DSS+Boswellic acid composition of Example 2: 41 mg/kg
Group 4—DSS+Boswellic acid composition of Example 4: 41 mg/kg
Group 5—DSS+Sulfasalazine: 30 mg/kg

Body weights were recorded daily throughout the study period. Macroscopic examination was carried out for all the terminal sacrificed, moribund and found dead animals. Colon and rectum were collected, and histopathology was performed.

The percentage of mortality during the study period was 0%, 20%, 0%, 0% and 20% for vehicle control group without DSS (G1), DSS positive group (G2), DSS+Test Item 1 (G3), DSS+Test Item 2 (G4) and DSS+Reference Item (G5), respectively. Dullness and hunched back posture were observed during the induction as well as treatment periods from Day 4 onwards and persisted up to Day 10.

Mean DAI in disease-controlled group was 3.1 in males and 3.9 in females on Day 10 (last day of induction period). In animals treated with Test Item 1 and Test Item 2, mean DAI was found to be decreased (3.2 and 2.8 for males; 2.8 and 2.2 for females, respectively) indicating recovery of symptoms in animals treated with Boswellia acid compositions when compared to sulfasalazine (reference; DAI was 3.6 and 3.2 in males and females, respectively) treated animals.

The % body weight was significantly decreased on a day to day basis in DSS induced colitis groups (G2, G3, G4 and G5) as compared with vehicle control (i.e. without DSS treatment) group. However, animals treated with Boswellic acid compositions exhibited lesser weight loss on Day 10 as compared to disease control as well as sufasalazine treated animals (−0.44%, −20.34%, −14.74%, −13.38% and −17.77% for G1, G2, G3, G4 and G5, respectively).

During macroscopic examination, colon with red mucosa was observed in most of the DSS treated animals. However, the severity of the lesion was found to be comparatively minimal in animals treated with boswellic acid compositions. The microscopic examination proved the induction of prominent colitis as observed in terminal colon and rectum of DSS positive control group, test and reference treatment groups when compared to vehicle control group. Erosion accompanied with regenerative changes such as inflammation of mucosa, submucosa and thickening of muscularis mucosa in terminal colon and rectum were observed in all DSS induced animals. Additional regenerative changes such as inflammatory cell infiltration with edema, epithelial cell regeneration, neovascularization and fibroblast proliferation were also observed. The observed regenerative lesions indicate better regeneration of damaged tissues in boswellic acid composition treated animals when compared to reference as well as disease control animals.

Conclusion: Based on the study results, it is concluded that boswellic acid compositions exhibited better gut-protection activity as compared to those animals treated with Sulfasalazine (30 mg/kg, G5) in DSS induced colitis model in BALB/C mice following 10 days repeated dose administration.

Evaluation of Gut-Protective Effect Through Antimicrobial Activity of Boswellic Acid Composition Against Helicobacter pylori Strains

Micro-broth dilution method was used to determine the antimicrobial activity of boswellic acid composition against Helicobacter pylori strains. The anti-bacterial minimum inhibitory concentration (MIC) study was performed as per the Clinical and Laboratory Standards Institute (CLSI) guidelines.

The test compound (composition comprising boswellic acid) was dissolved using Methanol to a concentration of 12.8 mg/mL (stock). Amoxicillin was dissolved in sterile Phosphate buffer to a concentration of 2 mg/mL. Test composition, and Amoxicillin (reference antibiotic) were tested at 128 μg/mL to 0.25 μg/mL MIC value can be considered as the concentration of compound at which the growth was inhibited to the extent of >90%, compared to growth control.

Results: Boswellic acid composition showed approximately 30% reduction of tester strain Helicobacter pylori at 128 μg/mL concentration. MIC of Amoxicillin against Helicobacter pylori was <0.25 μg/mL. The results indicate a definitive antimicrobial activity of boswellic acid composition against H. pylori.

Conclusion: The administration of boswellic acids composition indicated better regeneration of damaged tissues in process of combating colitis and maintaining gut-health. The compositions also indicated a definitive antimicrobial activity against H. pylori. Both the evaluations substantiated the clinically observed in gut-protective effect of boswellic acid composition.

1. A gut-protective composition exhibiting enhanced dissolution, comprising 5 to 95% boswellic acids,
at least one pH modifier, and
optionally one more excipient which is acceptable in nutraceutical, food and pharmaceutical industry.

2. The composition of claim 1, wherein boswellic acids may be comprised of α-boswellic acid, β-boswellic acid, γ-boswellic acid, its acetyl or keto derivative including 3-acetyl-β-boswellic acid, 11-keto-β-boswellic acid, 3-acetyl-α-boswellic acid, 3-acetyl-11-keto-β-boswellic acid, salt, an active metabolite, a polymorph, a solvate, a hydrate, an enantiomer, an optical isomer, a tautomer and a racemic mixture thereof.

3. The composition of claim 2, wherein the boswellic acids may be comprised of about 15% to about 70% by weight of acetyl-11-keto-β-boswellic acid.

4. The composition of claim 2, wherein the boswellic acids may be comprised of about 5% to about 40% by weight of β-boswellic acid.

5. The composition of claim 2, wherein the boswellic acids may be comprised of about 1% to about 20% by weight of 11-keto-β-boswellic acid.

6. The composition of claim 1, wherein pH modifier is selected from alkaline agents, amino sugars, alkali metal salts, alkaline earth metal salts, metal oxide, organic bases, organic basic salts, inorganic bases, inorganic basic salts, conjugate bases of weak organic acids and mixtures thereof.

7. The composition of claim 6, wherein pH modifier is selected from meglumine, N-methyl glucamine, magnesium oxide, aluminum oxide, magnesium hydroxide, calcium hydroxide, sodium hydroxide, ammonium hydroxide, sodium citrate dihydrate, sodium lauryl sulphate, sodium acetate, potassium hydroxide, aluminum hydroxide, potassium carbonate, sodium bicarbonate, calcium carbonate, calcium hydrogen phosphate, sodium phosphate, potassium phosphate, calcium phosphate, magnesium phosphate, ammonium phosphate and mixtures thereof.

8. The composition of claim 6, wherein the pH modifier is comprised from about 10 to 80% by weight of the composition.

9. The composition of claim 1, wherein the ratio of boswellic acids to pH modifier ranges from about 1:0.1 to 1:10.

10. The composition of claim 1, wherein optional excipient can be selected from diluents, binders, disintegrants, lubricants, glidants, surfactants, solubilizers, wetting agents, stabilizers, buffers, preservatives, sorbents, antioxidants, complexing agents, viscosity enhancers, plasticizers, coating materials, sweeteners, colors, flavors and the mixture thereof.

11. A process for preparation of gut-protective composition comprising boswellic acid, wherein the steps are comprised of
a. mixing boswellic acid and pH modifier by optionally adding one excipient
b. blending well to get a mixture
c. processing the mixture to get granular powder composition
